(19) United States
(12) Patent Application Publication
(10) Pub. No.: US 2020/0354402 A1
(43) Pub. Date: Nov. 12, 2020

(54) PEPTIDE COMPOSITIONS AND RELATED METHODS

(71) Applicant: Allegro Pharmaceuticals, LLC, San Juan Capistrano, CA (US)

(72) Inventors: John Y Park, Santa Ana, CA (US); Hampar L. Karageozian, San Juan Capistrano, CA (US); Vicken H. Karageozian, San Juan Capistrano, CA (US)

(21) Appl. No.: 16/882,660

(22) Filed: May 25, 2020

Related U.S. Application Data
(62) Division of application No. 16/012,706, filed on Jun. 19, 2018, now abandoned.
(60) Provisional application No. 62/521,984, filed on Jun. 19, 2017.

(51) Int. Cl. C07K 5/062; A61K 38/06; A61K 38/08; A61K 38/12; C07K 7/64; C07K 7/06; A61K 47/20; C07K 5/083; C07K 5/09; A61K 9/00; C07K 5/097; C07K 5/093; A61K 38/07; A61K 31/185; A61K 38/05; C07K 5/103; C07K 5/12

(52) U.S. Cl. CPC C07K 5/06026; C07K 5/12; A61K 38/08; A61K 38/12; C07K 7/64; C07K 7/06; A61K 47/20; C07K 5/081; C07K 5/0815; A61K 9/0048; C07K 5/0821; C07K 5/0819; C07K 5/0817; A61K 38/07; A61K 31/185; A61K 38/05; C07K 5/0806; C07K 5/1008; A61K 38/06

(57) ABSTRACT

Peptide compositions and methods for inhibiting neovascularization or development of pathological or aberrant blood vessels in human or other animal subjects.

### RELATED APPLICATION

This patent application is a division of U.S. patent application Ser. No. 16/012,706 filed Jun. 19, 2018, which claims priority to U.S. Provisional Patent Application No. 62/521,984 entitled Peptide Compositions and Related Methods filed Jun. 19, 2017, the entire disclosure of each such application being expressly incorporated herein by reference.

### FIELD OF THE INVENTION

The present invention relates generally to the fields of Biology and medicine and more particularly to peptide compositions and their methods of use.

### BACKGROUND

Pursuant to 37 CFR 1.71(e), this patent document contains material which is subject to copyright protection and the owner of this patent document reserves all copyright rights whatsoever.

Throughout this patent application amino acids may be referred to interchangeably using the following names, three letter codes and single letter codes:

| Amino Acid | Three letter code | Single Letter Code |
|---|---|---|
| Alanine | Ala | A |
| Arginine | Arg | R |
| Asparagine | Asn | N |
| Aspartic Acid | Asp | D |
| Cysteine | Cys | C |
| Cysteic Acid | Cys(Acid) | — |
| Glutamic | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tyrosine | Tyr | Y |
| Valine | Val | V |

Applicant is developing the synthetic oligopeptide Glycinyl-Arginyl-Glycinyl-Cysteic(Acid)-Threonyl-Proline (ALG-1001 or Luminate®, Allegro Ophthalmics, LLC) which has been shown to inhibit a number of integrins and to have significant antiangiogenic, anti-inflammatory, neuroprotective and other effects. When administered to the eye, ALG-1001 can cause vitreolysis, posterior vitreo-retinal detachment (PVD) and is useable for treatment of eye disorders such as wet macular degeneration (WMD), dry macular degeneration (DMD), diabetic retinopathy (PDR), diabetic macular edema (DME) and vitreomacular traction (VMT). Further information regarding ALG-1001 and related compounds is found in U.S. Pat. No. 9,018,352 entitled Peptide Compositions and Therapeutic Uses Thereof, U.S. Pat. No. 9,872,886 entitled Compositions and Methods for Inhibiting Cellular Adhesion or Directing Diagnostic or Therapeutic Agents to RGD Binding Sites and U.S. Pat. No. 9,896,480 entitled Integrin Receptor Antagonists and Their Methods of Use as well as pending U.S. patent application Ser. No. 15/874,814 entitled Therapeutic and Neuroprotective Peptides, the entire disclosure of each such patent and patent application being expressly incorporated herein by reference.

As described below, Applicant has synthesized and performed initial testing on a number of additional novel peptides, a number of which exhibit therapeutic effects in in vivo tests.

### SUMMARY

In accordance with the present invention, there are provided peptide compounds and methods for inhibiting neovascularization of the development of pathological or aberrant blood vessels in human or animal subjects.

In accordance with one aspect of the present invention, there are provided compositions of matter which comprise peptides that consist of or include an amino acid sequence having the formula:

Y—X—Z

wherein:
Y=R, H, K, Cys(acid), G or D;
X=G, A, Cys(acid), R, G, D or E; and
Z=Cys(acid), G, C, R, D, N or E.
   Such peptides may comprise or consist of the amino acid sequences; R-G-Cys(Acid), R-R-Cys, R-CysAcid)-G, Cys(Acid)-R-G, Cys(Acid)-G-R, R-G-D, R-G-Cys(Acid). H-G-Cys(Acid), R-G-N, D-G-R, R-D-G, R-A-E, K-G-D, R-G-Cys(Acid)-G-G-G-D-G, Cyclo-{R-G-Cys(acid)-F-N-Me-V}, R-A-Cys (Acid), R-G-C, K-G-D, Cys(acid)-R-G, Cys(Acid)-G-R, Cyclo-{R-G-D-D-F-NMe-V}, H-G-Cys(acid) and salts thereof. Possible salts include but are not limited to acetate, trifluoroacetate (TFA) and hydrochloride salts. Such peptides are useful at least for inhibiting neovascularization of the development of pathological or aberrant blood vessels in human or animal subjects

Further in accordance with the present invention, peptides of the present invention, or the synthetic oligopeptide Glycinyl-Arginyl-Glycinyl-Cysteic(Acid)-Threonyl-Proline, may be combined with Taurine and administered to a human or animal subject for the purpose of inhibiting neovascularization of the development of pathological or aberrant blood vessels

Still further in accordance with the present invention, there are provided methods for inhibiting neovascularization or the development of pathological or aberrant blood vessels in a human or animal subjects who are in need thereof, such methods comprising the step of administering to the subject a therapeutically effective amount of a composition comprising a peptide as summarized above. In some instances, such methods may be carried out to treat a disease or disorder of the eye wherein neovascularization or development of pathological or aberrant blood vessels occurs. Such diseases or disorders of the eye include but are not necessarily limited to: diabetic retinopathy, neovascular age-related macular degeneration, retinopathy of prematurity (ROP), sickle cell retinopathy, retinal vein occlusion, ischemia-induced retinopathy and certain inflammatory diseases of the eye.

Still further in accordance with the present invention, there are provided methods for inhibiting neovascularization or the development of pathological or aberrant blood vessels in human or animal subjects at locations outside of the eye. In some instances, such methods may be carried out to inhibit the growth or metastasis of a vascularized tumor.

Still further aspects and details of the present invention will be understood upon reading of the detailed description and examples set forth herebelow.

### BRIEF DESCRIPTION OF THE DRAWINGS

FIG. 1 is a bar graph of retinal neovascular area in ROP mouse eyes following treatment with either His-Gly-Cys(acid) (Test Compound No. 14) or Control Peptide (Arg-Gly-Glu).

FIG. 2 is a bar graph of retinal neovascular area in ROP mouse eyes following treatment with either Arg-Gly-Cys(acid)TFA (Positive Control), Arg-Ala-Cys (Test Compound No. 3) or Control Peptide (Arg-Gly-Glu).

FIG. 3 is a bar graph of retinal neovascular area in ROP mouse eyes following treatment with either Arg-Gly-Cys(acid)TFA (Test Compound No. 1/positive control), Arg-Ala-Asp (Test Compound No. 23) or Control Peptide (Arg-Gly-Glu).

FIG. 4 is a bar graph of retinal neovascular area in ROP mouse eyes following treatment with either Arg-Gly-Cys(acid)TFA (Positive Control), Arg-Ala-Cys(Acid) (Test Compound No. 3) or Control Peptide (Arg-Gly-Glu).

FIG. 5 is a bar graph of retinal neovascular area in ROP mouse eyes following treatment with either Arg-Gly-Cys(acid)TFA (Positive Control), Arg-Gly-Cys (Test Compound No. 4) or Control Peptide (Arg-Gly-Glu).

FIG. 6 is a bar graph of retinal neovascular area in ROP mouse eyes following treatment with either Arg-Gly-Cys(acid)TFA (Positive Control), Arg-Gly-Cys(acid)TFA (Masked) (Test Compound No. 1) or Control Peptide (Arg-Gly-Glu).

FIG. 7 is a bar graph of retinal neovascular area in ROP mouse eyes following treatment with either Lys-Glys-Asp (Test Compound No. 20) or Control Peptide (Arg-Gly-Glu).

FIG. 8 is a bar graph of retinal neovascular area in ROP mouse eyes following treatment with either His-Gly-Cys(Acid) (Test Compound No. 14) or Control Peptide (Arg-Gly-Glu).

FIG. 9 is a bar graph of retinal neovascular area in ROP mouse eyes following treatment with either Lys-Gly-Cys(acid) (Test Compound No. 6) or Control Peptide (Arg-Gly-Glu).

FIG. 10 is a bar graph of retinal neovascular area in ROP mouse eyes following treatment with either Arg-Gly-Cys(acid)TFA (Positive Control), Arg-Cys(Acid)-Gly (Test Compound No. 5) or Control Peptide (Arg-Gly-Glu).

FIG. 11 is a bar graph of retinal neovascular area in CNV mouse eyes following treatment with either Arg-Gly-Cys(acid)TFA (Positive Control), Arg-Gly-Cys(Acid) Acetate (Test Compound No. 2) or Control Peptide (Arg-Gly-Glu).

FIG. 12 is a bar graph of retinal neovascular area in ROP mouse eyes following treatment with either Arg-Gly-Cys(acid)TFA (Positive Control), Arg-Gly-Cys(Acid) Acetate (Test Compound No. 2) or Control Peptide (Arg-Gly-Glu).

FIG. 13 is a bar graph of retinal neovascular area in ROP mouse eyes following treatment with either Asp-Gly-Arg (Test Compound No. 17) or Control Peptide (Arg-Gly-Glu).

FIG. 14 is a bar graph of retinal neovascular area in ROP mouse eyes following treatment with either Arg-Gly-Asp (Test Compound No. 15) or Control Peptide (Arg-Gly-Glu).

FIG. 15 is a bar graph of retinal neovascular area in ROP mouse eyes following treatment with either Arg-Cys(Acid)-Gly (Test Compound No. 18) or Control Peptide (Arg-Gly-Glu).

FIG. 16 is a bar graph of retinal neovascular area in ROP mouse eyes following treatment with either Arg-Gly-Cys(acid)-Gly-Gly-Asp-Gly (Test Compound No. 7) or Control Peptide (Arg-Gly-Glu).

FIG. 17 is a bar graph of retinal neovascular area in ROP mouse eyes following treatment with either Arg-Ala-Glu (Test Compound No. 19) or Control Peptide (Arg-Gly-Glu).

FIG. 18 is a bar graph of retinal neovascular area in ROP mouse eyes following treatment with either Gly-Cys(acid)-Arg (Test Compound No. 11) or Control Peptide (Arg-Gly-Glu).

FIG. 19 is a bar graph of retinal neovascular area in ROP mouse eyes following treatment with either Cys(Acid)-Ala-Arg (Test Compound No. 10) or Control Peptide (Arg-Gly-Glu).

FIG. 20 is a bar graph of retinal neovascular area in ROP mouse eyes following treatment with either Arg-Glu-Gly (Test Compound No. 22) or Control Peptide (Arg-Gly-Glu).

FIG. 21 is a bar graph of retinal neovascular area in ROP mouse eyes following treatment with either Cys(acid)-Arg-Gly (Test Compound No. 8) or Control Peptide (Arg-Gly-Glu).

FIG. 22 is a bar graph of retinal neovascular area in ROP mouse eyes following treatment with either Arg-Gly-Asn (Test Compound No. 16) or Control Peptide (Arg-Gly-Glu).

FIG. 23 is a bar graph of retinal neovascular area in ROP mouse eyes following treatment with either Arg-Gly-Cys(acid)TFA (Positive Control), Cyclo-{R-G-D-D-F-NMe-V} (Test Compound No. 13) or Control Peptide (Arg-Gly-Glu).

FIG. 24 is a bar graph of retinal neovascular area in ROP mouse eyes following treatment with either Arg-Gly-Cys(acid)TFA (Positive Control), Cyclo-{R-G-Cys(acid)-F-N-Me-V} (Test Compound No. 12) or Control Peptide (Arg-Gly-Glu).

FIG. 25 is a bar graph of retinal neovascular area in ROP mouse eyes following treatment with either Arg-Gly-Cys(acid)TFA (Positive Control), Cys(Acid)-Gly-Arg (Test Compound No. 9) or Control Peptide (Arg-Gly-Glu).

FIG. 26 is a bar graph of retinal neovascular area in ROP mouse eyes following treatment with either His-Gly-Cys(Acid) (Test Compound No. 14) or Control Peptide (Arg-Gly-Glu).

FIG. 27 is a bar graph of retinal neovascular area in ROP mouse eyes following treatment with either Arg-Gly-Cys(acid)TFA (Positive Control), Taurine (Test Compound No. 25), Arg-Gly-Cys(acid). TFA+Taurine (Test Compound No. 24) or Control Peptide (Arg-Gly-Glu).

### DETAILED DESCRIPTION

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

A number of diseases and disorders are known to cause neovascularization or development of pathological or aberrant blood vessels, including diabetic retinopathy, neovascular age-related macular degeneration, retinopathy of prematurity (ROP), sickle cell retinopathy, retinal vein occlusion, ischemia-induced retinopathy, certain inflammatory diseases of the eye and the growth or metastasis of a vascularized tumors. Applicant has discovered a number of compounds that are shown to be active in an anti-neovascularization mouse ROP model as described below. On this basis, such compounds are potentially useful in the treatment of diseases and disorders which are known to cause neovascularization or development of pathological or aberrant blood vessels, including but not limited to those diseases and disorders listed above.

Each test compound was prepared in sterile water for injection, containing 0.08 mg/100 μL of sodium chloride and 0.005 mg/100 μL of trisodium citrate, the concentration of the peptide was at a concentration of 2.0 mg/100 μL and pH=2.7 and dispensed by sterile filtration into sterile vials. The Taurine test compound was obtained from Sigma Aldrich company, which was >99% pure, and prepared the same way as mentioned previously, having a concentration of 3.0 mg/100 μL. The R-G-Cys(acid) at 2.0 mg/100 μL+Taurine at 3.0 mg/100 μL were prepared the same way as mentioned above

To screen the test compounds for activity against ischemia-induced retinal neovascularization, the well-established model of retinopathy of prematurity (ROP) in mice was used. Litters of C57BI/6 mice were placed in 75% oxygen at postnatal day (P) 7, returned to room air at day (P) 12. The Pups were randomly assigned to treatment groups of 4 to 10 animals per group. The pups were treated as follows: Treatment eyes were treated by intravitreal injection of 1.0 microliters of solution containing 20 micrograms of Test Compound.

On post-natal day 17, 5 days after intravitreal injection, the animals were sacrificed, the retinas were flat mounted and the area of neovascularization in each retina was determined by Fluorescein-dextran image analysis.

Applicant has identified the tripeptide R-G-Cysteic(Acid) as an integrin binding motif of the oligopeptide Glycinyl-Arginyl-Glycinyl-Cysteic(Acid)-Threonyl-Proline (ALG-1001 or Luminate®, Allegro Ophthalmics, LLC). The trifluoroacetate (TFA) and acetate salts of the R-G-Cysteic(Acid) tripeptide (Test Compound Nos. 1 and 2) were tested in both the ROP Mouse Model as described above as well as in a mouse model of choroidal neovascularization induced by laser photocoagulation (“CNV Mouse Model”), as generally as described in Lambert, V., et al., Laser-Induced Choroidal Neovascarization Model to Study Age Related Macular Degeneration in Mice, Nature Protocols, 8; 2197-2211 (2013). Animals assigned to “Control” groups were treated by intravitreal injection of Arg-Gly-Glu (Control Peptide), which is known to be inactive. In some of the experiments, an additional “Positive Control” group was included. Animals assigned to a “Positive Control” group were treated by intravitreal injection of Arg-Gly-Cys(acid)TFA, which is known to be active.

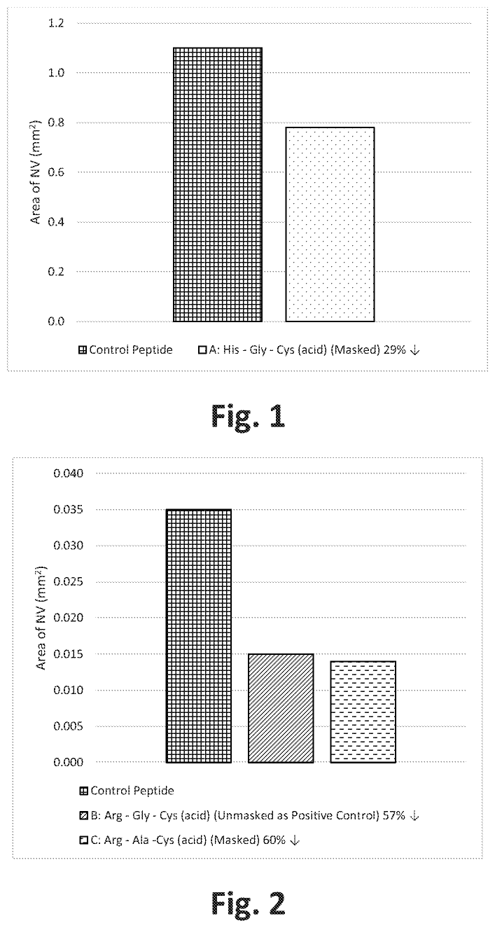

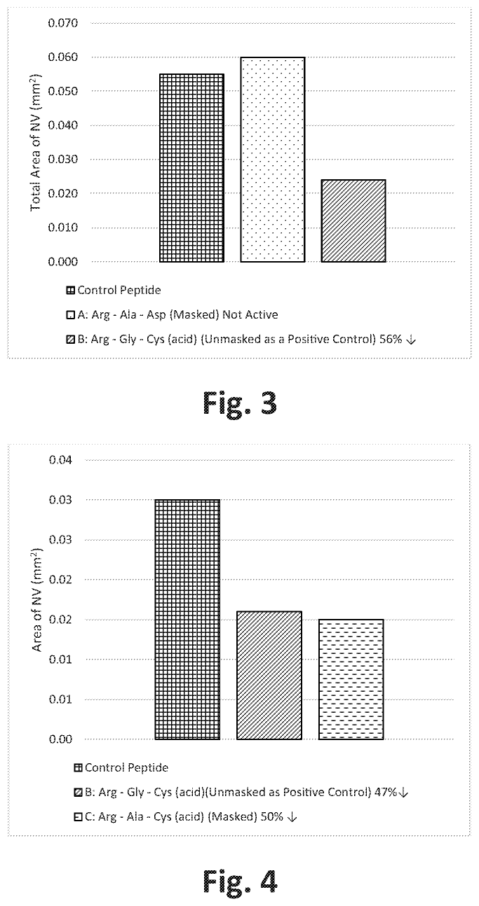

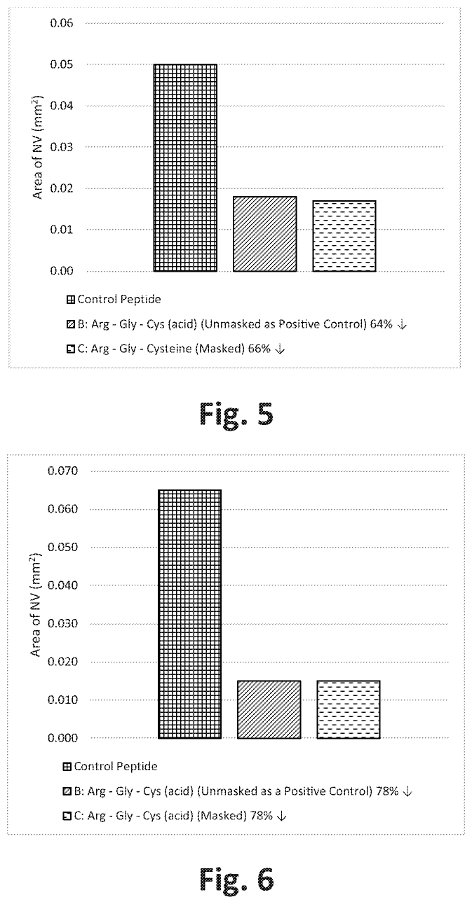

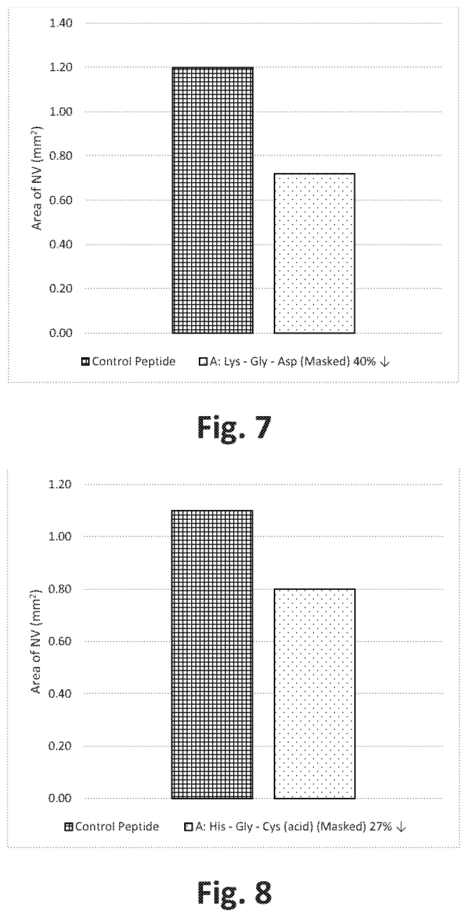

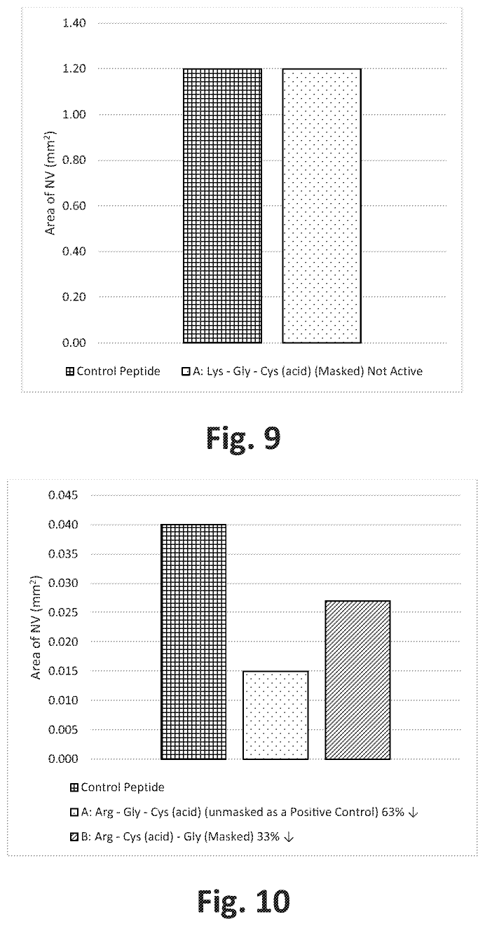

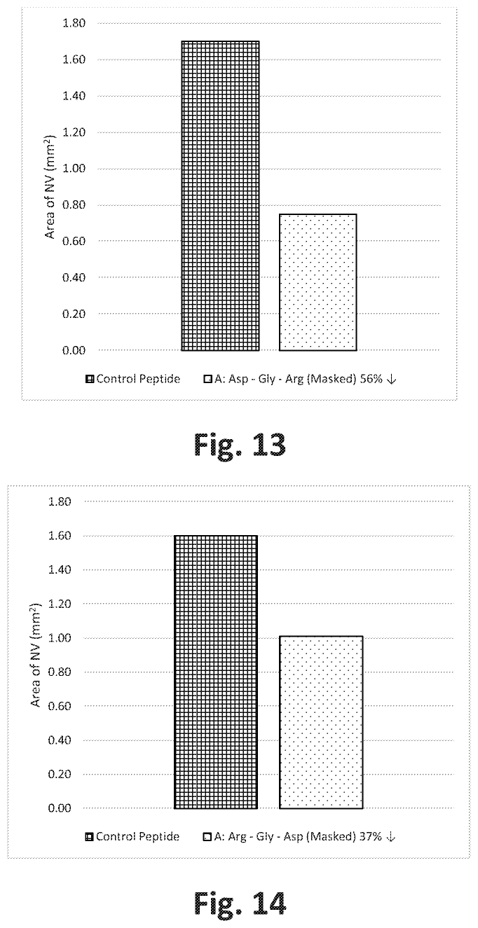

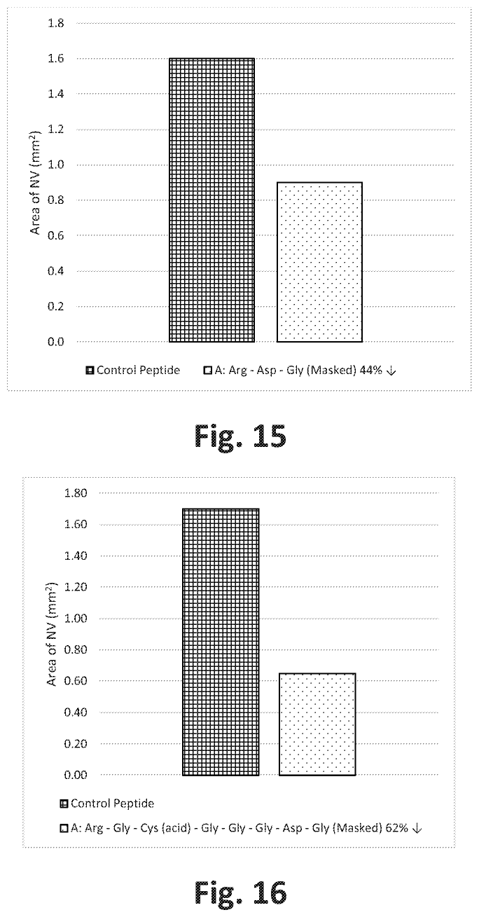

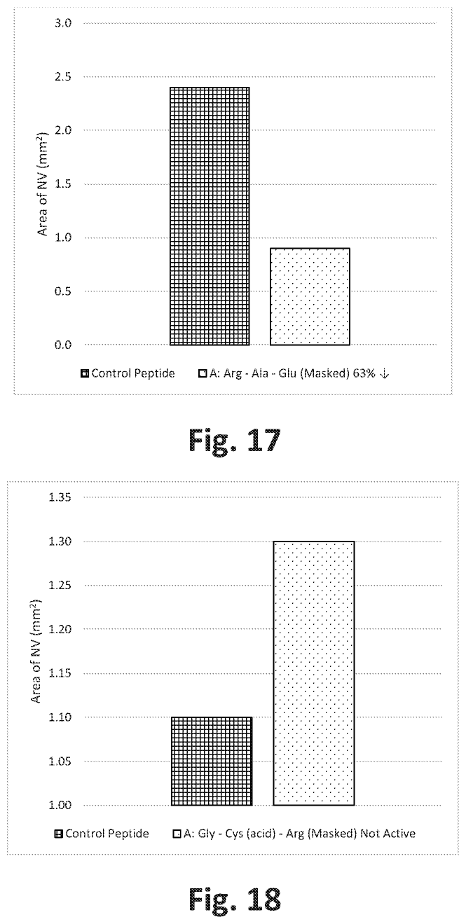

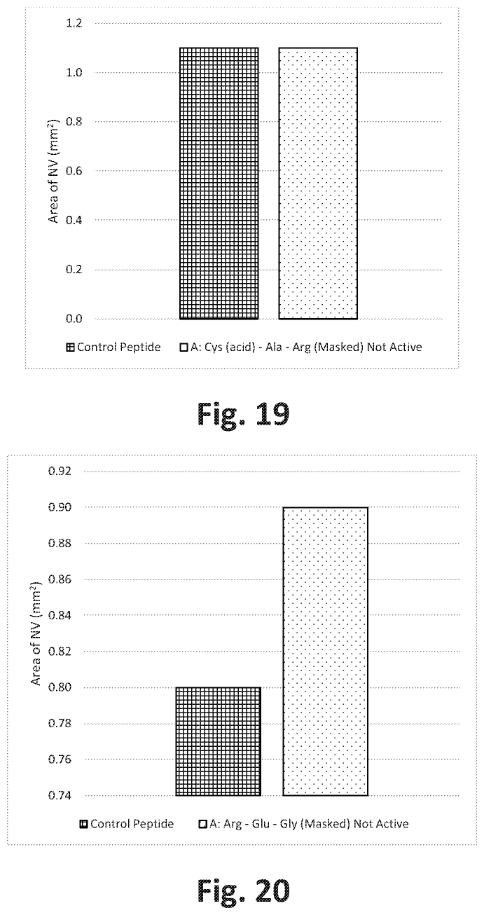

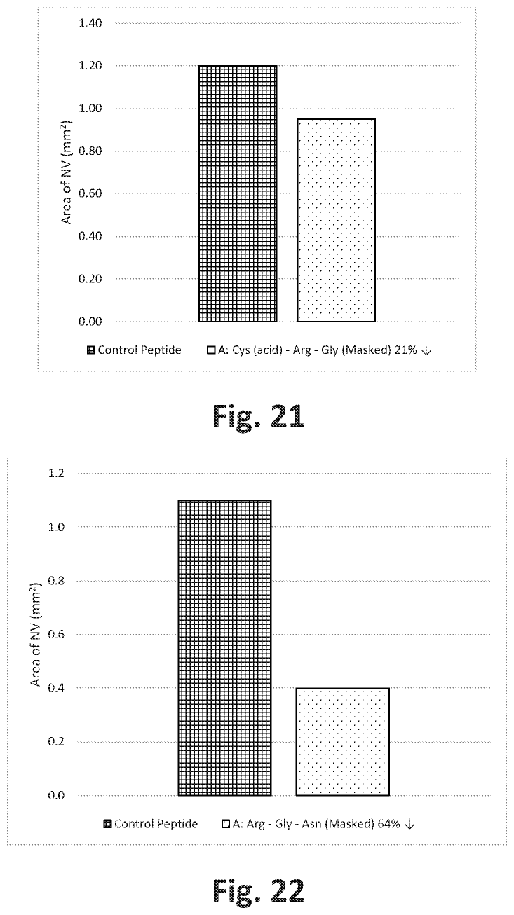

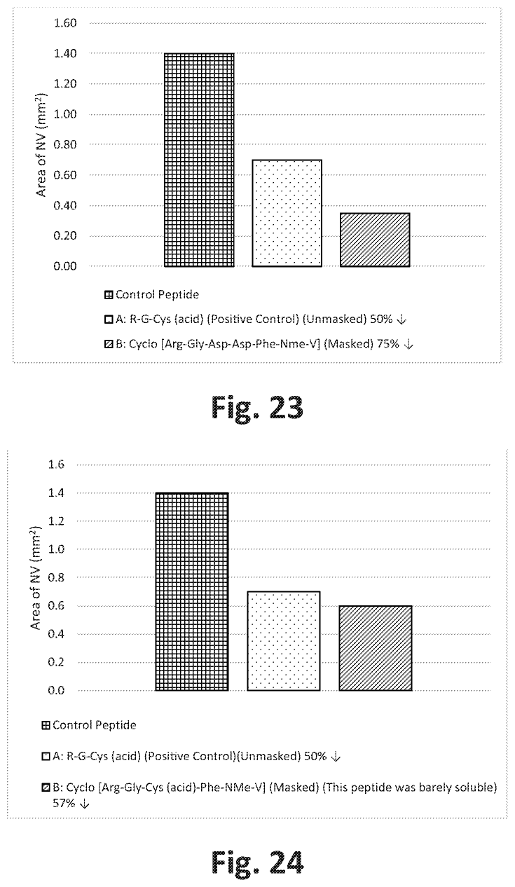

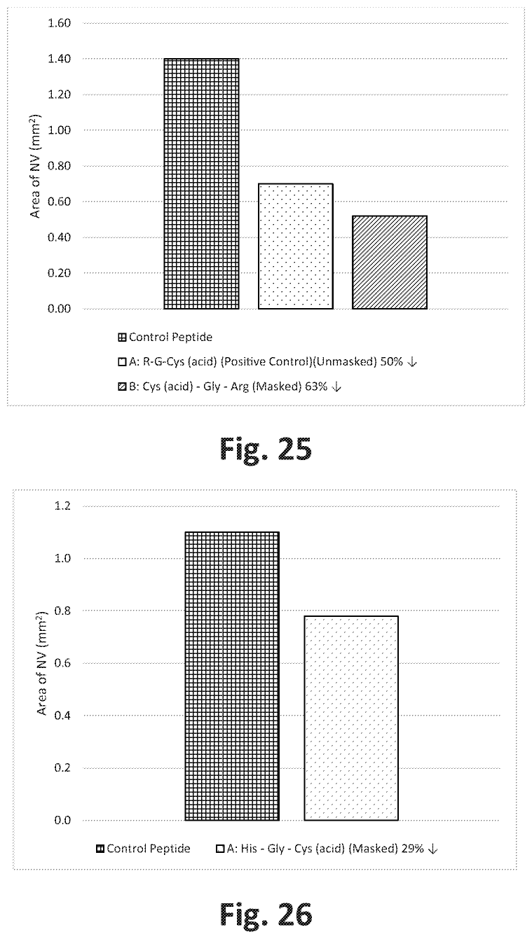

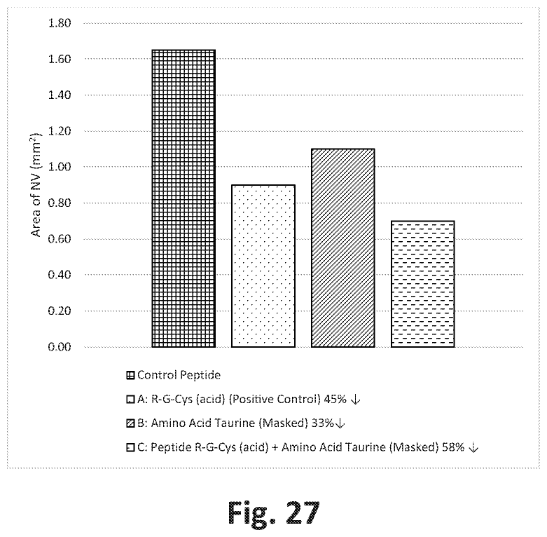

The following Table 1 summarizes the neovascularization inhibiting effect of each Test Compound at the dose tested. In each instance, the data was obtained using the ROP Mouse Model, except for the two table entries specifically labeled “CNV”. Only those table entries labeled “CNV” show data obtained from the CNV Mouse Model. Bar graphs showing the test results summarized in Table 1 are also provided herewith as FIGS. 1 through 27. Where indicated in the figures, the tests were performed in a blinded manner such that the persons performing the testing did not know the identity or structure of each test compound.

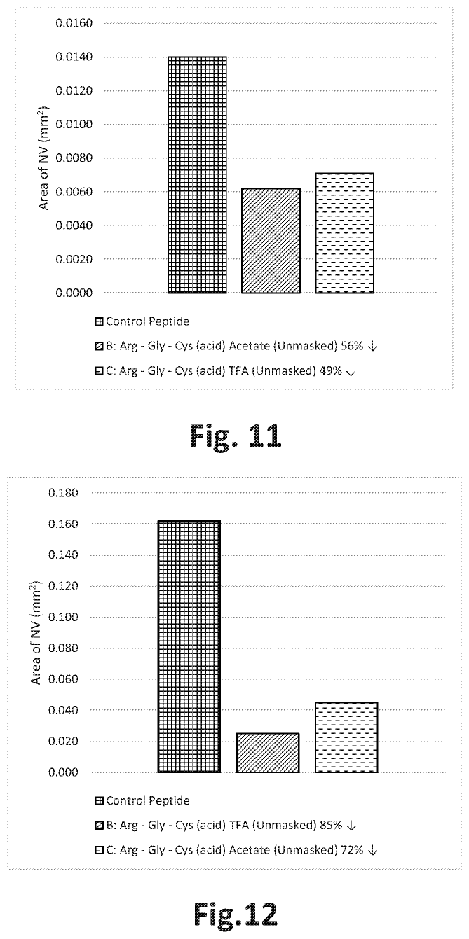

TABLE 1
SUPPRESSION OF RETINAL NEOVASCULARIZATION IN MOUSE MODEL
OF ROP (ISCHEMIC) RETINOPATHY

| | | Mean % Reduction | |
|---|---|---|---|
| Test | | of Retinal | |
| Compound | | Neovascularization | Activity |
| Number | Test Compound | In ROP Model | At Dose Tested |
| 1 | R—G-Cys(acid).TFA-ROP | 61 | Active |
| 1(CNV) | R—G-Cys(acid).TFA-CNV | 49-FIG. 11 | Active |
| 2(CNV) | R—G-Cys(acid).Acetate-CNV | 56-FIG. 11 | Active |
| 2 | R—G-Cys(acid).Acetate-ROP | 72 | Active |
| 3 | R—A-Cys (acid).TFA | 60 | Active |
| 4 | R—G-Cysteine.TFA | 66 | Active |
| 5 | R-Cys(acid)—G.TFA | 33 | Slightly Active |
| 6 | K—G-Cys(acid).TFA | 0 | Not Active |
| 7 | R—G-Cys(acid)—G—G—G—D—G.TFA | 62 | Active |
| 8 | Cys(acid)—R—G.TFA | 21 | Slightly Active |
| 9 | Cys(acid)—G—R.TFA | 63 | Active |
| 10 | Cys(acid)—A—R.TFA | 0 | Not Active |
| 11 | G-Cys(acid)—R.TFA | 0 | Not Active |
| 12 | Cyclo-{R—G-Cys(acid)—F—N—Me— | 57 | Active |
| | V}Acetate | | |
| 13 | Cyclo-{R—G—D—D—F—NMe—V}.TFA | 75 | Active |
| 14 | H—G-Cys(acid).TFA | 28 | Slightly Active |
| 15 | R—G—D.TFA | 37 | Slightly Active |
| 16 | R—G—N.TFA | 64 | Active |
| 17 | D—G—R.TFA | 56 | Active |
| 18 | R—D—G.TFA | 44 | Active |
| 19 | R—A—E.TFA | 63 | Active |
| 20 | K—G—D.TFA | 40 | Active |
| 21 | R—G—E.TFA | 0 | Not Active |
| 22 | R—E—G.TFA | 0 | Not Active |
| 23 | R—A—D.TFA | 0 | Not Active |
| 24 | R—G-Cys(acid).TFA + Taurine | 58 | Active |
| 25 | Taurine | 33 | Slightly Active |

In some of the Test Compounds, the amino acid sequence of the binding motif RGCys(acid) tripeptide in GRGCys(acid)TP (ALG-1001) was rearranged and/or replaced by other basic, acidic and neutral amino acids. Based on the results of the ROP and CNV testing summarized above, the result indicates that the presence of Arginine, Alanine and Cysteic Acid in the GRGCys(acid)TP peptide (ALG-1001/Luminate) plays an important role in the suppression of the neovascularization, notably the sequence of R-G-Cys and R-A-Cys. Furthermore, in the presence of arginine, replacement of Cysteic (Acid) by a neutral amino acid exhibited a strong suppressive effect in these experiments.

Y—X—Z General Formula 1

Wherein:
Y=R*, H, K, Cys(acid), G or D;
X=G*, A, Cys(acid), R, G, D or E; and
Z=Cys*, G, Cysteine, R, D, N or E.
*indicates component of the RGCys(acid) binding motif of tripeptide in GRGCys(acid)TP (ALG-1001), which was used as a Positive Control.

Based on the initial data set forth herein, certain structure/activity relationships are suggested in relation to specific changes made to the R-G-Cysteic Acid binding motif. For example, when the amino acid R (i.e., the Y Component) of the R-G-Cysteic(Acid) binding motif is replaced by a basic amino acid or acidic amino acid, the peptide's anti-neovascularization effects diminish, whereas in the presence of arginine in the binding motif aspartic acid as Component Y appears to promote the peptide's anti-neovascularization effects.

When amino acid G (i.e., the X Component) of the R-G-Cysteic Acid binding motif is replaced by a basic or acidic amino acid, the peptide's anti-neovascularization effects decrease. However, in the presence of arginine (a strong hydrogen bonding), two carbon length-space for hydrophobic interaction (Alanine and Aspartic Acid) may not influence the peptide's anti-neovascularization effects.

When Cys(Acid) (i.e., the Z Component) of the R-G-Cysteic(Acid) binding motif is replaced by a neutral amino acid, the peptide's neovascularization inhibiting activity increases whereas replacement of the Z component by acidic or basic amino acids causes the neovascularization inhibiting activity to decrease.

All indications are that the R-G-Cysteic(Acid) of the oligopeptide Glycinyl-Arginyl-Glycinyl-Cysteic(Acid)-Threonyl-Proline (ALG-1001 or Luminate®, Allegro Ophthalmics, LLC) is important for suppression of neovascularization. Also, addition of three parts taurine to one part of the Glycinyl-Arginyl-Glycinyl-Cysteic(Acid)-Threonyl-Proline (ALG-1001) improves the neovascularization suppressing activity.

It is to be appreciated that, although the invention has been described hereabove with reference to certain examples or embodiments of the invention, various additions, deletions, alterations and modifications may be made to those described examples and embodiments without departing from the intended spirit and scope of the invention. For example, any elements, steps, members, components, compositions, reactants, parts or portions of one embodiment or example may be incorporated into or used with another embodiment or example, unless otherwise specified or unless doing so would render that embodiment or example unsuitable for its intended use. Also, where the steps of a method or process have been described or listed in a particular order, the order of such steps may be changed unless otherwise specified or unless doing so would render the method or process unsuitable for its intended purpose. Additionally, the elements, steps, members, components, compositions, reactants, parts or portions of any invention or example described herein may optionally exist or be utilized in the absence or substantial absence of any other element, step, member, component, composition, reactant, part or portion unless otherwise noted. All reasonable additions, deletions, modifications and alterations are to be considered equivalents of the described examples and embodiments and are to be included within the scope of the following claims.

What is claimed is:

1.-23. (canceled)

24. A method for inhibiting neovascularization or the development of pathological or aberrant blood vessels in a human or animal subject who is in need thereof, said method comprising the step of administering to the subject a therapeutically effective amount of a composition comprising a peptide which consists of or includes an amino acid sequence having the formula
Y—X—Z
wherein:
Y=R, H, K, Cys(acid), G or D;
X=G, A, Cys(acid), R, G, D or E; and
Z=Cys(acid), G, C, R, D, N or E.

25. A method according to claim 24 wherein the composition further comprises taurine.

26. A method according to claim 24 wherein the method is carried out to inhibit neovascularization or the development of pathological or aberrant blood vessels in the eye.

27. A method according to claim 26 wherein the method is carried out to treat a disease or disorder selected from: diabetic retinopathy, neovascular age-related macular degeneration, retinopathy of prematurity (ROP), sickle cell retinopathy, retinal vein occlusion, ischemia-induced retinopathy and inflammatory diseases of the eye.

28. A method according to claim 24 wherein the method is carried out to inhibit neovascularization or the development of pathological or aberrant blood vessels outside of the eye.

29. A method according to claim 28 wherein the method is carried out to inhibit the growth or metastasis of a vascularized tumor.

30. A method according to claim 24 wherein the peptide comprises R-G-Cys(acid)-G-G-G-D-G.

31. A method according to claim 24 wherein the peptide comprises Cyclo-{R-G-Cys(acid)-F-N-Me-V}.

32. A method according to claim 24 wherein the amino acid sequence is R-A-Cys (acid).

33. A method according to claim 24 wherein the amino acid sequence is Cys(acid)-G-R.

34. A method according to claim 24 wherein the peptide comprises Cyclo-{R-G-D-D-F-NMe-V}.

35. A method according to claim 24 wherein the amino acid sequence is H-G-Cys(acid).

36. A method according to claim 24 wherein the amino acid sequence is R-G-N.

37. A method according to claim 24 wherein the amino acid sequence is D-G-R.

38. A method according to claim 24 wherein the amino acid sequence is R-D-G.

39. A method according to claim 24 wherein the amino acid sequence is R-A-E.

40. A method according to claim 24 wherein the amino acid sequence is K-G-D.
